(19) United States
(12) Patent Application Publication
(10) Pub. No.: US 2006/0235545 A1
(43) Pub. Date: Oct. 19, 2006

(54) Prosthetic foot with fully adjustable hindfoot and forefoot keels and inversion/eversion, pronation/supination capability

(76) Inventor: Matthew Joseph Habecker, Bellevue, MI (US)

Correspondence Address: Matthew J. Habecker, 9445 Jones Road, Bellevue, MI, 49021, US

(21) Appl. No.: 11/388,631

(22) Filed: Mar. 24, 2006

Related U.S. Application Data
(60) Provisional application No. 60/670,966, filed on Apr. 13, 2005.

(51) Int. Cl. A61F 2/66

(52) U.S. Cl. 623/55; 623/53

(57) ABSTRACT

A prosthetic foot possessing a forefoot keel cluster (1) which is comprised of three distinct keels. The keels are functionally connected to one another by way of an anterior pitched glider (6) which is positioned above the two lower keels and beneath the upper keel. The anterior pitched glider (6) is tapped and houses an anterior screw (7) which acts as a worm gear to reposition the glider with respect to the forementioned keels of the forefoot keel cluster (1). The position of the glider determines the functional lengths of the two lower keels and their associated deflection properties. A rearfoot keel cluster (2) also exists with a posterior pitched glider (14) and posterior screw (15) that serves a matching function to that of the anterior pitched glider (6) found in the forefoot keel cluster (1). The rearfoot keel cluster (2) is functionally connected to the forefoot keel cluster (1) by way of a keel divider (10). The entire prosthetic foot can be attached to standard prosthetic componentry by way of an endoskeletal adapter (18) found on top of the keel divider (10).

### BACKGROUND

### Description of Prior Art

Prosthetic feet have assumed many forms over the years. The more recent embodiments such as U.S. Pat. No. 4,547,913 to Phillips have been constructed from space-age composites which are strong, light-weight and possess spring-like properties. The forefoot and hindfoot sections of these feet are designed to deflect in a certain way at specific stages of the gait cycle to reproduce a cosmetic gait pattern. These dynamic response feet are usually created by the manufacturer with deflection properties determined by an individual patient's height, weight and activity level. Once these prosthetic feet have been fabricated, little can be done to adjust their performance characteristics in clinic. If the foot seems too stiff or too soft to a patient after alignment adjustments have been made, the foot must be re-manufactured until the desired walking characteristics are obtained.

There remains a terrific need for a prosthetic foot whose keels may be individually adjusted to optimize a patient's walking abilities.

### SUMMARY

In accordance with the present invention a prosthetic foot comprises triangulated anterior and posterior keel clusters, joined together by a keel divider, and dynamically connected by their respective pitched gliders which are moved in space by position screws.

### OBJECTS AND ADVANTAGES

Accordingly, besides the objects and advantages of the prosthetic ankle joint described in my above patent, several objects and advantages of the present invention are:
a) To provide a prosthetic foot whose forefoot keel deflection properties may be independently adjusted in clinic to optimize the characteristics of the foot through mid to late stance.
b) To provide a prosthetic foot whose hindfoot keel deflection properties may be independently adjusted in clinic to optimize the characteristics of the foot through initial contact to mid stance.
c) To provide a prosthetic foot that allows for inversion/eversion capability at any selected hindfoot keel resistance level.
d) To provide a prosthetic foot that allows for supination/pronation capability at any selected forefoot keel resistance level.
e) To provide a prosthetic foot that is light weight, durable and possesses a minimum number of moving parts.
f) To provide a prosthetic foot that requires little, if any, maintenance.
g) To provide a prosthetic foot that allows for rapid and convenient adjustment by the prosthetist or user.
h) To provide a prosthetic foot that allows for effective multiaxial motion without mechanical axes.
i) To provide a prosthetic foot that allows for attachment to standard endoskeletal prosthetic componentry.
j) To provide a prosthetic foot that may be housed within a cosmetic foot-shell covering.
k) To provide a prosthetic foot which is appropriate for any level or classification of patient walking ability.
l) To provide a prosthetic foot which can be cheaply manufactured and provided to a greater patient population due to its affordability.

### DRAWING FIGURES

FIG. 1 is an isometric view of the prosthetic foot.

FIG. 2 is a side view of the prosthetic foot.

FIG. 3 is a top view of the prosthetic foot.

### REFERENCE NUMERALS IN DRAWINGS

1 Forefoot keel cluster
2 Rearfoot keel cluster
3 Medial anterior cluster keel
4 Lateral anterior cluster keel
5 Dorsal anterior cluster keel
6 Anterior pitched glider
7 Anterior screw
8 Anterior screw retainer
9 Anterior screw retainer nut
10 Keel cluster divider
11 Medial posterior cluster keel
12 Lateral posterior cluster keel
13 Dorsal posterior cluster keel
14 Posterior pitched glider
15 Posterior screw
16 Posterior screw retainer
17 Posterior screw retainer nut
18 Endo skeletal adapter

### DESCRIPTION—FIGS

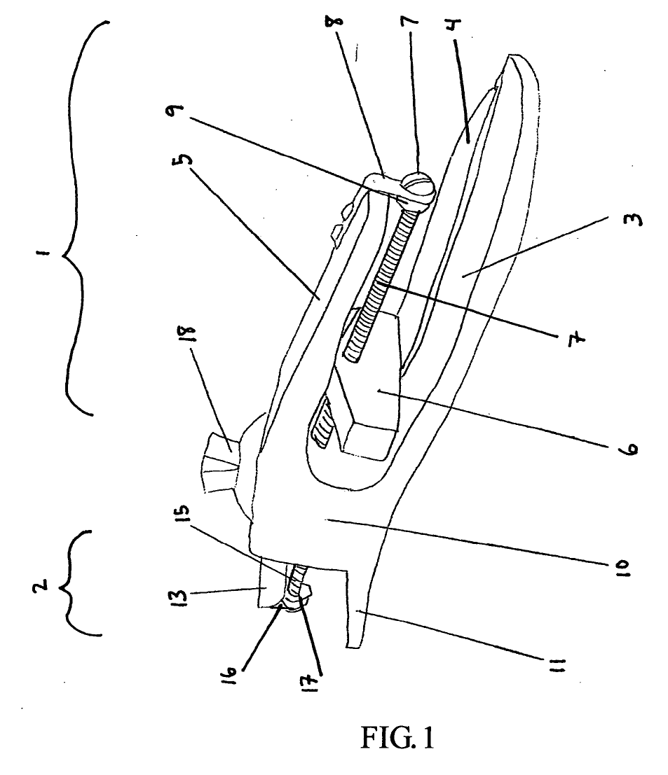

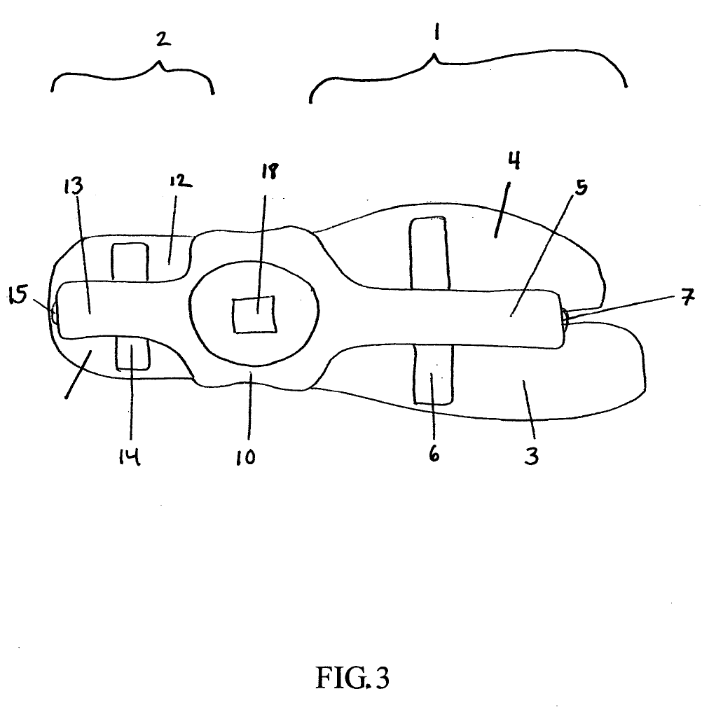

A preferred embodiment of the prosthetic foot is illustrated in FIG. 1-3. In the preferred embodiment, the foot would be comprised of light-weight composites or plastics. The present embodiment is comprised of a forefoot keel cluster 1 and rearfoot keel cluster 2. The forefoot keel cluster 1 is subdivided into a medial anterior cluster keel 3, lateral anterior cluster keel 4 and dorsal anterior cluster keel 5. The rearfoot keel cluster 2 of the foot is also subdivided into a medial posterior cluster keel 11, lateral posterior cluster keel 12 and dorsal posterior cluster keel 13. All anterior and posterior keels mentioned originate from the keel cluster divider 10. The dorsal anterior cluster keel 5 and dorsal posterior cluster keel 13 are centrally situated above their respective medial and lateral cluster keels.

The forefoot keel cluster 1 keels are dynamically connected by an anterior pitched glider 6 whose proximal apex makes contact with the central, underside of the dorsal anterior cluster keel 5. The lateral and medial base of the anterior pitched glider 6 makes contact with the respective lateral anterior and medial anterior cluster keels 4,3. The anterior pitched glider 6 is tapped and positioned in space between the fore-mentioned anterior keels by a threaded anterior screw 7. The anterior screw 7 is secured to the end of the dorsal anterior cluster keel 5 by way of the anterior screw retainer 8 and anterior screw retainer nut 9. The opposite end of the anterior screw 7 is free to rotate within the keel cluster divider 10 to act as a worm gear within the prosthetic foot. When rotated, the anterior screw 7 causes the anterior pitched glider 6 to translate between the dorsal anterior cluster keel 5 and the lateral and medial anterior cluster keels 4,3.

In an identical fashion, the hindfoot keel cluster 2 keels are dynamically connected by a posterior pitched glider 14 whose proximal apex makes contact with the central, underside of the dorsal posterior cluster keel 13. The lateral and medial base of the posterior pitched glider 14 makes contact with the respective lateral posterior and medial posterior cluster keels 12,11. The posterior pitched glider 14 is tapped and positioned in space between the fore-mentioned posterior keels by a threaded posterior screw 15. The posterior screw 15 is secured to the end of the dorsal posterior cluster keel 13 by way of the posterior screw retainer 16 and posterior screw retainer nut 17. The opposite end of the posterior screw 15 is free to rotate within the keel cluster divider 10 to act as a worm gear within the prosthetic foot. When rotated, the posterior screw 15 causes the posterior pitched glider 14 to translate between the dorsal posterior cluster keel 13 and the lateral and medial posterior cluster keels 12,11.

The prosthetic foot may be connected to industry-standard prosthetic componentry by way of its endoskeletal adapter 18 which is secured to the keel cluster divider 10.

### ADVANTAGES

From the description above, a number of advantages of my prosthetic foot become evident:
(a) The deflection characteristics of the medial and lateral anterior cluster keels may be changed by the position of the anterior pitched glider above them.
(b) When walking on uneven terrain, the sloped top and flat bottom of the anterior pitched glider allows it to pivot on the underside of the dorsal anterior cluster keel and maintain contact with the medial and lateral anterior cluster keels, respectively.
(c) The deflection characteristics of the medial and lateral posterior cluster keels may be changed by the position of the posterior pitched glider above them.,
(d) When walking on uneven terrain, the sloped top and flat bottom of the posterior pitched glider allows it to pivot on the underside of the dorsal posterior cluster keel and maintain contact with the medial and lateral posterior cluster keels, respectively.
(e) The existence of distinct forefoot and hindfoot keel clusters allows for independent adjustment according to the unique needs encountered during the various phases of the gait cycle.
(f) The minimum number of moving parts allows the foot to assume a lightweight, low-maintenance embodiment.
(g) The simple, worm-gear design, allows for rapid and convenient adjustments to be made by a practitioner or patient.

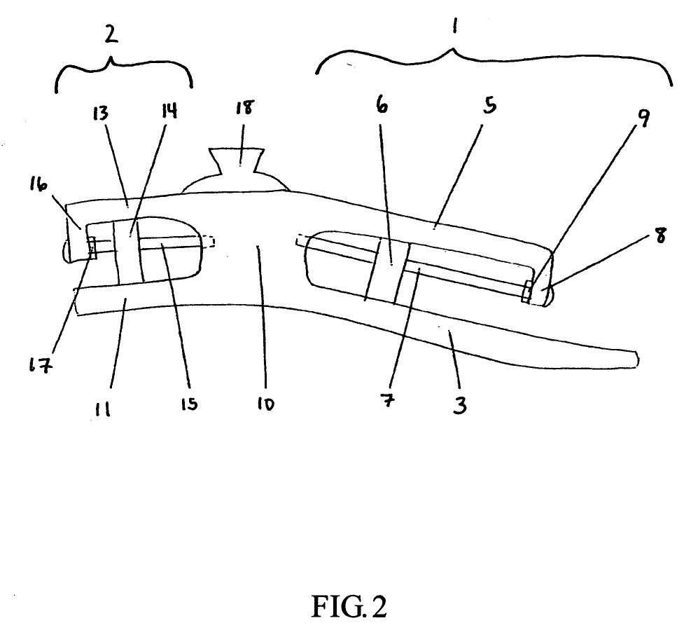

### OPERATION—FIGS 1, 2,3

The forces from initial contact and loading response cause the medial and lateral posterior cluster keels 11,12 to deflect upwards. The functional length of these keels is determined by the position of the posterior pitched glider 14 above them. The posterior pitched glider 14 controls the functional lengths of the keels beneath itself by directing a portion of the impact forces to the more rigid dorsal posterior cluster keel 13. The sloped upper surfaces of the posterior pitched glider 14 and its apex's pseudo-articulation with the dorsal posterior cluster keel 13, allow for uneven deflections of the associated medial and lateral posterior cluster keels 11,12 on its undersurface.

As the amputee's weight shifts over the prosthetic foot, the forefoot keel cluster 1 starts to engage. In a similar fashion, the functional length of these anterior keels are determined by the position of the anterior pitched glider 6 above them. The anterior pitched glider 6 controls the functional lengths of the keels beneath itself by directing a portion of the impact forces to the more rigid dorsal anterior cluster keel 5. The sloped upper surfaces of the anterior pitched glider 6 and its apex's pseudo-articulation with the dorsal anterior cluster keel 5, allow for uneven deflections of the associated medial and lateral anterior cluster keels 3,4 on its undersurface.

By independently controlling the functional lengths of the appropriate forefoot and rearfoot keel clusters 1,2, the deflection properties of the foot may be precisely adjusted for specific events of the gait cycle. The shapes of the pitched gliders allow for a preservation of the desired keel lengths while allowing for inversion/eversion, supination/pronation motions of the foot. The pitched glider positions may be controlled through the adjustment of their associated anterior and posterior screws 7,15. The anterior screw 7 is secured to the dorsal anterior cluster keel 5 by way of an anterior screw retainer 8 and anterior screw retainer nut 9. The posterior screw 15 is secured to the dorsal posterior cluster keel 13 by way of a posterior screw retainer 16 and posterior screw retainer nut 17. Clockwise rotation of these screws will translate their respective pitched gliders away from the keel cluster divider 10. The prosthetic foot may be connected to industry standard prosthetic componentry by way of its endoskeletal adapter 18.

This adjustment capability is currently unavailable in current prosthetic feet and would afford amputees and practitioners an unprecedented level of component customization. Ultimately, the characteristics of the prosthetic foot could be fine-tuned to save energy, optimize gait, and increase the functional abilities of its user.

### CONCLUSION, RAMIFICATIONS AND SCOPE

Although previous dynamic response prosthetic foot designs have sought to employ the latest use of materials and technology, they have failed to make provisions for significant customization of foot performance. As mentioned earlier, these composite foot keel resistance levels are manufactured according to formulas which are based on various patient measurements and walking categories. Even though the manufacturers of such feet would claim that they are custom made for individual patients, once the feet are fabricated, their fundamental walking characteristics are unalterable.

By allowing practitioners or patients to adjust the forefoot and hindfoot keel properties of their prosthetic foot, a new level of customization is possible. A fine-tuned hindfoot keel would allow for comfortable shock absorption at initial contact and allow the foot to transition nicely to midstance. The fine-tuned forefoot keel would wallow for a perfect balance of stiffeness for amputees as they walk over their feet in mid to late stance. By altering the effective toe lever of the foot, the step lengths and general walking symmetry of amputees can be optimized.

The preservation of inversion/eversion, supination/pronation capability in the foot allows for conformance of its plantar surface to uneven terrain even as the functional keel lengths are changed. This feature translates into additional stability for amputees as they encounter various environments. Since each prosthesis is painstakingly fit to its individual user, it is felt that the fundamental components used in its construction should also allow for a high degree of customization. Although prosthetic technology has advanced to a significant degree over the years, true customization of dynamic response feet is not yet available. The proposed design seeks to fill this critically important void in the prosthetic component landscape.

I claim:

1. A prosthetic foot with adjustable forefoot and hindfoot keels and inversion/eversion, supination/pronation capability comprising:
a forefoot keel cluster;
a hindfoot keel cluster;
a keel cluster divider for joining proximal prosthetic components;
adjustable means to control forefoot keel stance phase dorsiflexion;
adjustable means to control hindfoot keel stance phase plantarflexion;
means for hindfoot to conform to uneven terrain through effective inversion and eversion motion;
means for forefoot to conform to uneven terrain through effective supination and pronation motions.

2. The prosthetic foot of claim 1 wherein said forefoot keel cluster is comprised of a medial anterior cluster keel, a lateral anterior cluster keel and a dorsal anterior cluster keel.

3. The prosthetic foot of claim 2 wherein said medial anterior cluster keel is a flat, cantilevered extension of said keel cluster divider and comprises a fraction of the foot's plantar surface.

4. The prosthetic foot of claim 2 wherein said lateral anterior cluster keel is a flat, cantilevered extension of said keel cluster divider, comprises a fraction of the foot's plantar surface and is slightly shorter than said medial anterior cluster keel.

5. The prosthetic foot of claim 2 wherein said dorsal anterior cluster keel is a flat, cantilevered extension of said keel cluster divider and is positioned above and between said medial anterior cluster keel and said lateral anterior cluster keel.

6. The prosthetic foot of claim 2 wherein said dorsal anterior cluster keel undersurface is flat and parallel to the top surfaces of said medial anterior cluster keel and said lateral anterior cluster keel.

7. The prosthetic foot of claim 2 wherein said dorsal anterior cluster keel has an integrated anterior screw retainer.

8. The prosthetic foot of claim 7 wherein said anterior screw retainer has an aperature of sufficient size to accommodate an anterior screw.

9. The prosthetic foot of claim 8 wherein said anterior screw passes through said anterior screw retainer, is threaded through said anterior pitched glider and is secured at its end by an aperature of a matching radius within said keel cluster divider.

10. The prosthetic foot of claim 8 wherein said anterior screw is free to rotate relative to said anterior screw retainer but is held in place by an anterior screw retainer nut.

11. The prosthetic foot of claim 10 wherein said anterior screw retainer nut is on the opposite side of said anterior screw retainer as that of the head of said anterior screw and is semi-permanently attached to said anterior screw.

12. The prosthetic foot of claim 2 wherein said forefoot keel cluster houses an anterior pitched glider which is positioned between said dorsal anterior cluster keel, said medial anterior cluster keel and said lateral anterior cluster keel.

13. The prosthetic foot of claim 12 wherein said anterior pitched glider has a top with two sloped surfaces whose apex makes tangential contact with the underside of said dorsal anterior cluster keel in the medial lateral plane and is perpendicular to the long axis of the dorsal anterior cluster keel.

14. The prosthetic foot of claim 12 wherein said anterior pitched glider has a mildly radiused base which makes tangential contact with the top surfaces of said medial anterior anterior cluster keel and said lateral anterior cluster keel in the anterior posterior plane and is perpendicular to the long axes of said medial anterior cluster keel and said lateral anterior cluster keel.

15. The prosthetic foot of claim 12 wherein said anterior pitched glider is tapped to accept the threads of said anterior screw.

16. The prosthetic foot of claim 1 wherein said hindfoot keel cluster is comprised of a medial posterior cluster keel, lateral posterior cluster keel and dorsal posterior cluster keel.

17. The prosthetic foot of claim 16 wherein said medial posterior cluster keel is a flat, cantilevered extension of said keel cluster divider and comprises a fraction of the foot's plantar surface.

18. The prosthetic foot of claim 16 wherein said lateral posterior cluster keel is a flat, cantilevered extension of said keel cluster divider and comprises a fraction of the foot's plantar surface.

19. The prosthetic foot of claim 16 wherein said dorsal posterior cluster keel is a flat, cantilevered extension of said keel cluster divider and is positioned above and between said medial posterior cluster keel and said lateral posterior cluster keel.

20. The prosthetic foot of claim 16 wherein said dorsal posterior cluster keel undersurface is flat and parallel to the top surfaces of said medial posterior cluster keel and said lateral posterior cluster keel.

21. The prosthetic foot of claim 16 wherein said dorsal posterior cluster keel has an integrated posterior screw retainer.

22. The prosthetic foot of claim 21 wherein said posterior screw retainer has an aperature of sufficient size to accommodate a posterior screw.

23. The prosthetic foot of claim 22 wherein said posterior screw passes through said posterior screw retainer, is threaded through said posterior pitched glider and is secured at its end by an aperature of a matching radius within said keel cluster divider.

24. The prosthetic foot of claim 22 wherein said posterior screw is free to rotate relative to said posterior screw retainer but is held in place by a posterior screw retainer nut.

25. The prosthetic foot of claim 24 wherein said posterior screw retainer nut is on the opposite side of said posterior screw retainer as that of the head of said posterior screw and is semi-permanently attached to said posterior screw.

26. The prosthetic foot of claim 16 wherein said hindfoot keel cluster houses a posterior pitched glider which is positioned between said dorsal posterior cluster keel, said medial posterior cluster keel and said lateral posterior cluster keel.

27. The prosthetic foot of claim 26 wherein said posterior pitched glider has a top with two sloped surfaces whose apex makes tangential contact with the underside of said dorsal posterior cluster keel in the medial lateral plane and is perpendicular to the long axis of the dorsal posterior cluster keel.

28. The prosthetic foot of claim 26 wherein said posterior pitched glider has a mildly radiused base which makes tangential contact with the top surfaces of said medial posterior cluster keel and said lateral posterior cluster keel in the anterior posterior plane and is perpendicular to the long axes of said medial posterior cluster keel and said lateral posterior cluster keel.

29. The prosthetic foot of claim 26 wherein said posterior pitched glider is tapped to accept the threads of said posterior screw.

30. The prosthetic foot of claim 1 wherein said forefoot keel cluster and said hindfoot keel cluster are joined by said keel cluster divider.

31. The prosthetic foot of claim 1 wherein the top of said keel divider houses an endoskeletal adapter to allow for connection of compatible endoskeletal prosthetic components.
